(19) United States
(12) Patent Application Publication
(10) Pub. No.: US 2022/0347076 A1
(43) Pub. Date: Nov. 3, 2022

(54) COSMETIC COMPOSITIONS COMPRISING CHITOSAN AND HYDROXYPROPYL METHYLCELLULOSE AND THEIR USE FOR IMPROVING HAIR STRENGTH AND RELATED PROPERTIES

(71) Applicant: Living Proof, Inc., Boston, MA (US)

(72) Inventors: Lauren A. Trahan, Boston, MA (US); Elizabeth May Patel, Boston, MA (US); Ronald Patrick McLaughlin, Boston, MA (US); Joseph Muscat, Boston, MA (US); Andrew R. Avery, Boston, MA (US)

(21) Appl. No.: 17/765,908

(22) PCT Filed: Oct. 5, 2020
(86) PCT No.: PCT/US2020/054281

§ 371 Date: Apr. 1, 2022

Related U.S. Application Data
(60) Provisional application No. 62/910,827, filed on Oct. 4, 2019.

(51) Int. Cl. A61K 8/73; A61Q 5/00

(52) U.S. Cl. CPC A61K 8/736; A61K 8/731; A61Q 5/002; A61K 2800/594

(57) ABSTRACT

Provided are hair treatment compositions comprising one or more polysaccharides and their use in improving hair strength.

### RELATED APPLICATIONS

This application claims priority to U.S. provisional application No. 62/910,827, filed Oct. 4, 2019, the entire contents of which are incorporated herein by reference.

### BACKGROUND

Healthy, undamaged hair is characterized by a cortex of closely packed cells surrounded by layers of overlapping, keratin rich cuticle cells, with a hydrophobic outer layer that is predominantly 18-methyl eicosanoic acid. Washing, drying, heat styling, and chemical services remove this hydrophobic exterior and leave the cuticle more susceptible to lifting and increasing surface friction. Repeated exposure to such conditions can further lift the cuticular cells—in some cases, completely removing them—resulting in a reduction in hair strength and, ultimately, breakage.

Curly hair has a number of structural characteristics that are different from straight hair and make it more prone to breakage. The internal structure of the hair follicle is modified, which results in an elliptical section of the hair fiber, as well as twists and bends in the hair fiber as it emerges from the follicle at the scalp. This ellipticity combined with the bends along the fiber make it sensitive to breakage from combing or brushing. Sebum typically lubricates the hair fiber, aiding in detangling. But, the morphology of curly hair fiber limits migration of sebum along the hair shaft. This only increases susceptibility to breakage when styling curly hair.

In addition to these biological characteristics, the styling habits of curly hair consumers also contribute to reduced fiber strength. Many curl consumers braid their wet hair to help it set into a more desirable curl pattern. This repeated breaking—especially when pulled too tight—further weakens both the hair fiber as well as the bulb. Thermal and chemical services intended to smooth or straighten the hair also result in a weakened hair fiber.

Traditional approaches to improving hair strength include the topical application of products that contain, e.g., proteins, emollients, cationic surfactants, and/or polymers. Unfortunately, each of these approaches have drawbacks. For example, while proteins and amino acids have been shown to reduce cuticular damage and minimize fiber breakage in damaged hair, consumers report that their hair is left feeling dry and brittle. Additionally, proteins are often challenging to formulate with, specifically as they contribute color and odor changes. Emollients including natural oils or butters (e.g., Argan oil or shea butter) and silicone oils improve hair surface lubricity. This typically results in fewer broken fibers when repeatedly brushing/styling the hair. However, the use of emollients tends to weigh hair down and leave an oily/greasy appearance.

Similar to emollients, cationic surfactants and cationic polymers are used to reduce the surface friction of the hair fiber, making it easier to brush. Many of these materials have been shown to reduce the number of broken fibers when repeatedly brushing the hair. However, the inherent positive ionic charge of cationic surfactants limits its compatibility with certain anionic systems like shampoos. Additionally, many cationic materials have been shown to build-up on hair with prolonged use.

Improved methods for increasing the strength and /or flexibility of hair while preserving a natural feel remains an unmet need.

### SUMMARY

It has now been found that compositions comprising one or more polysaccharides can improve the strength of hair. See e.g. the Exemplification section below. Provided, therefore, are compositions comprising one or more polysaccharides and their use in improving the strength of hair. Such polysaccharides include e.g., one or more of chitosan, hydroxylpropyl methylcellulose (HPMC), pectin, pullulan, and hyaluronic acid.

### BRIEF DESCRIPTION OF THE DRAWINGS

FIG. 1 is a comparison of the film strength, humidity resistance, and/or curl snap between various polysaccharides.

FIG. 2 is a comparison of the film strength, humidity resistance, and/or curl snap between various polysaccharides.

FIG. 3 is a comparison of the film strength, humidity resistance, and/or curl snap between various polysaccharides.

FIG. 4 is a comparison of the film strength of various polysaccharides.

FIG. 5 is a graph quantifying the difference in number of lifted cuticles on virgin hair swatches versus bleach hair fibers when exposed to increasing levels of strain.

FIG. 6. is a series of four images of untreated bleached hair fibers exposed to increasing levels of strain. Images were captured using the Sensofar S Neox model light microscope at 50× magnification.

FIG. 7. is a series of four images of bleached hair fibers, treated with an aqueous polysaccharide solution, and exposed to increasing levels of strain. Images were captured using the Sensofar S Neox model light microscope at 50× magnification.

FIG. 8. is a graph quantifying the difference in the number of lifted cuticles between the untreated bleached hair fibers and the bleached hair fibers treated with an aqueous polysaccharide solution when exposed to increasing levels of strain.

FIG. 9. is an image of the Lifecycle Rig setup used to repeatedly brush hair fibers.

FIG. 10. Is a graph quantifying the difference in broken fibers between untreated bleached hair and bleached hair treated with the inventive shampoo and conditioner.

### DETAILED DESCRIPTION

### 1. Compositions

Provided herein are hair treatment compositions comprising one or more polysaccharides selected from chitosan, hydroxylpropyl methylcellulose (HPMC), pectin, pullulan, and hyaluronic acid.

In a first aspect, the hair treatment compositions described herein comprise chitosan and HPMC.

In a second aspect, the hair treatment compositions described herein comprise chitosan and HPMC, wherein the ratio of chitosan to HPMC ranges from about 0.5 wt % to about 1.5 wt % chitosan to about 1.5 wt % to about 2.5 wt % HMPC. Alternatively, the ratio of chitosan to HPMC ranges from about 0.7 wt % to about 1.3 wt % chitosan to about 1.7 wt % to about 2.3 wt % HMPC; from about 0.8 wt % to about 1.2 wt % chitosan to about 1.8 wt % to about 2.2 wt % HMPC; or from about 0.9 wt % to about 1.1 wt % chitosan to about 1.9 wt % to about 2.1 wt % HMPC. In another aspect, the ratio of chitosan to HPMC is about 1.0 wt % chitosan to about 2.0 wt % HMPC.

In a third aspect, the chitosan in the disclosed compositions (e.g., as in the second aspect) is present in an amount of less than or equal to about 0.5 wt %, less than or equal to about 0.4 wt %, less than or equal to about 0.3 wt %, less than or equal to about 0.2 wt %, or less than or equal to about 0.1 wt % based on the total weight of the composition. Alternatively, as part of a second aspect, the chitosan in the disclosed compositions (e.g., as in the second aspect) is present in an amount ranging from about 0.01 wt % to about 0.5 wt %, from about 0.03 wt % to about 0.4 wt %, or from about 0.04 wt % to about 0.3 wt % based on the total weight of the composition.

In a fourth aspect, the HPMC in the disclosed compositions (e.g., as in the second or third aspect) is present in an amount of less than or equal to about 1 wt %, less than or equal to about 0.8 wt %, less than or equal to about 0.6 wt %, or less than or equal to about 0.4 wt %. Alternatively, as part of a third aspect, the HPMC in the disclosed compositions (e.g., as in the second or third aspect) is present in an amount ranging from about 0.05 wt % to about 0.6 wt %, from about 0.08 wt % to about 0.5 wt %, or from about 0.09 wt % to about 0.45 wt % based on the total weight of the composition.

In a fifth aspect, the disclosed compositions (e.g., as in the first through fourth aspects) further comprise a cosmetically acceptable medium. Alternatively, as part of a fifth aspect, the disclosed compositions (e.g., as in the first through third aspects) further comprise a cosmetically acceptable aqueous medium. In one aspect, this aqueous medium comprises at least one product having a water content of 40% or greater. In another aspect, this aqueous medium comprises at least one product having a water content of 40% or greater and can be applied to the hair for the purpose of cleansing, conditioning, or styling the hair (e.g., shampoo, rinse-off conditioner, leave-on conditioner, styling product).

In a sixth aspect, the disclosed compositions (e.g., as in the first through fifth aspects) further comprise one or more additional ingredients selected from anionic surfactants, nonionic surfactants, amphoteric surfactants, cationic surfactants, fatty alcohols, fatty acids, emulsifiers, emollients (e.g., silicones, hydrocarbons, esters, ethers, waxes, naturally derived oils and butters, and ceramides), glycols (e.g., glycerin, butylene glycol, propylene glycol, propanediol, pentylene glycol, and dipropylene glycol), polymers or rheology modifiers (e.g., gums, including but not limited to: guar, xanthan, tara, acacia or derivatives; cellulosics, including but not limited to: hydroxyethylcellulose, carboxymethylcellulose, hydroxypropylcellulose, or derivatives; and acrylic polymers, including but not limited to: carbomer, polyacrylamide, acrylates copolymer, acrylates/C10-30 Alkyl Acrylate Crosspolymer, polyacrylate crosspolymer-6, or similar), pH adjusters (including but not limited to: acids/pH decreasing agents (e.g., citric acid, lactic acid, glycolic acid, malic acid, gluconolactone or salts thereof) and bases/pH increasing agents (e.g., sodium hydroxide, potassium hydroxide, or amines, including, but not limited to: arginine, monoethanolamine, triethanolamine, triisopropanolamine, aminomethyl propanol, dimethylamino methylpropanol, tromethamine), UV absorbers, fragrance or other odor masking agents, and preservatives.

Specific compositions are described in the Exemplification section and are included herein.

### 2. Methods of Use

In one aspect, the disclosed compositions improve the strength of hair. In another aspect, the disclosed compositions are useful for improving the strength of hair.

### EXEMPLIFICATION

### Example 1. Methods for Determining Performance

The properties of various polysaccharides were evaluated as follows.

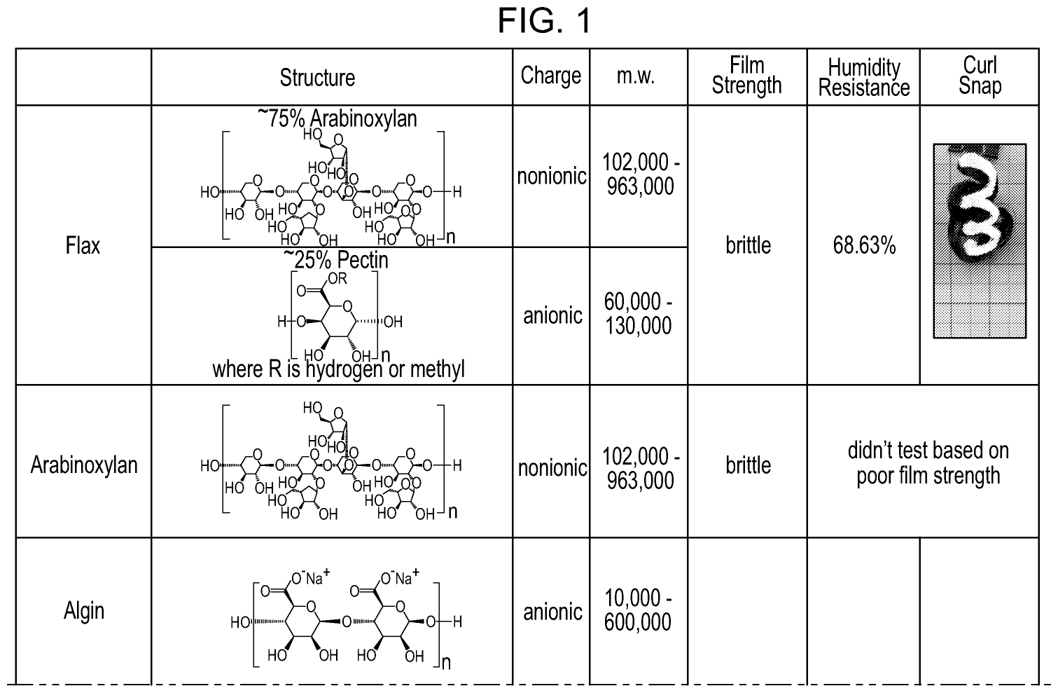

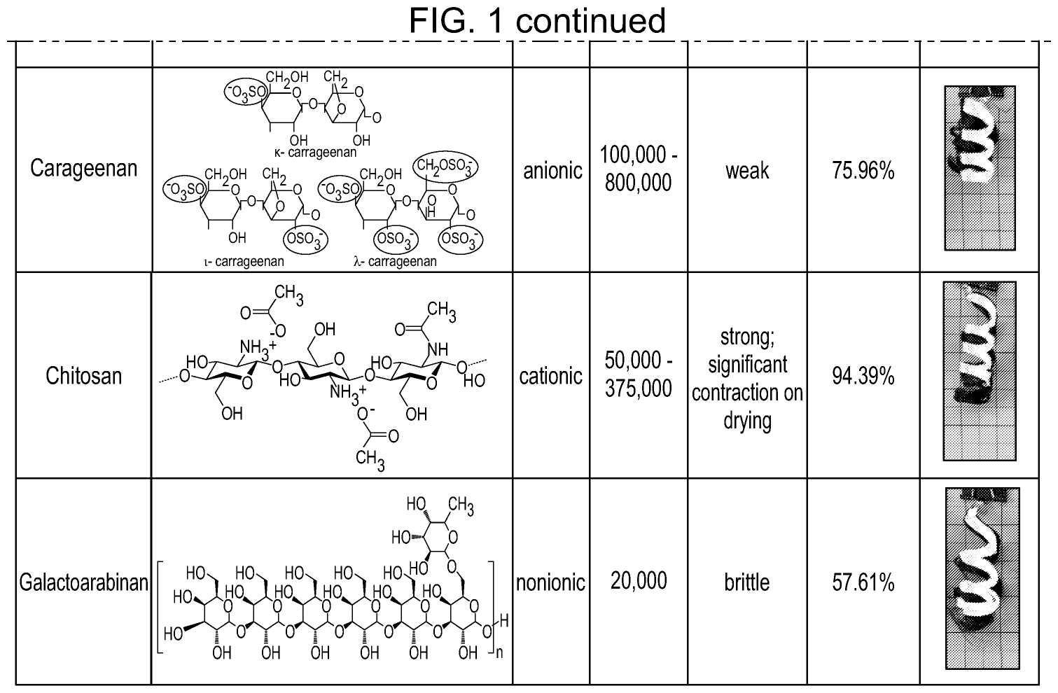

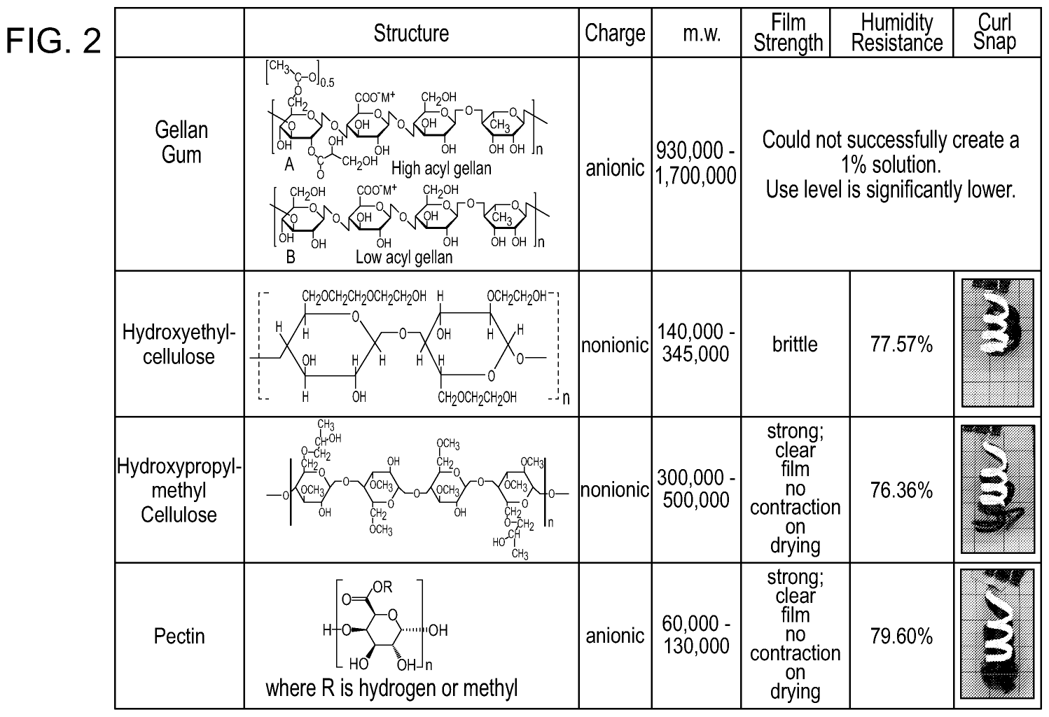

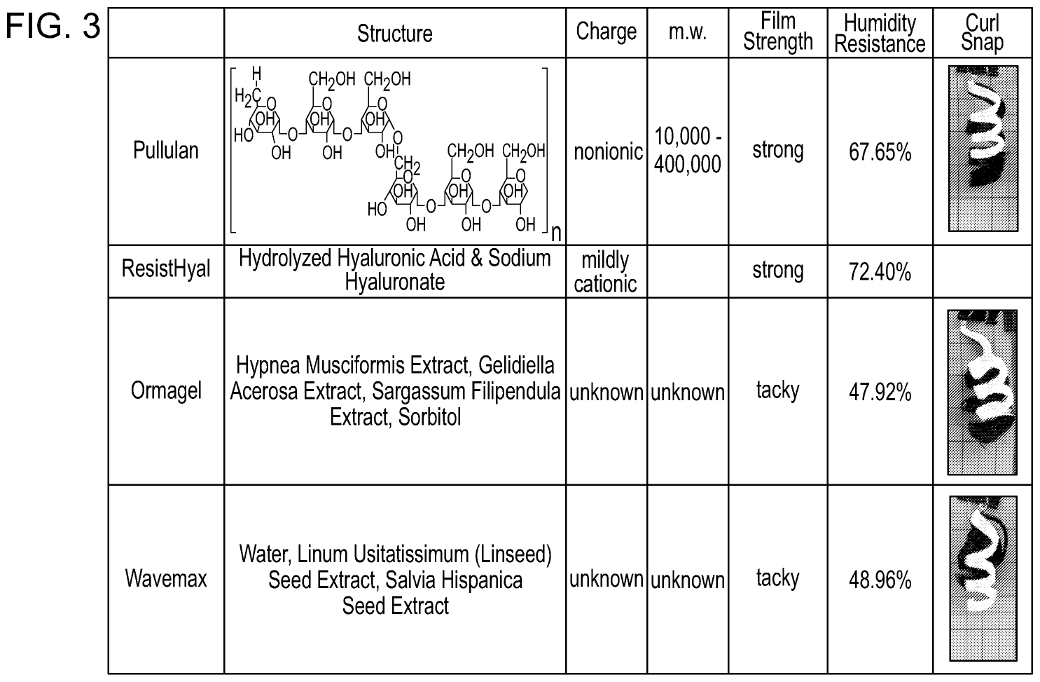

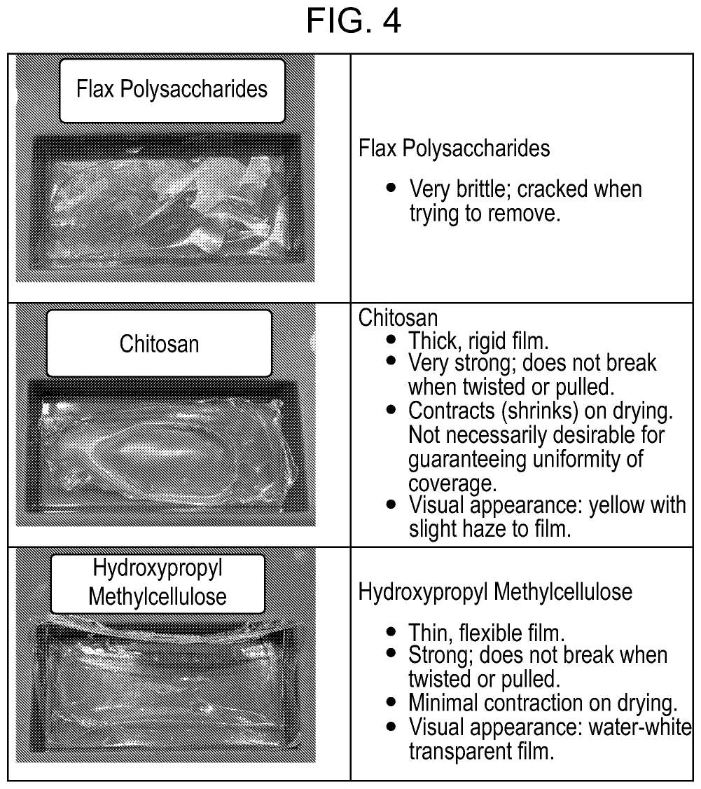

A 1% aqueous solution was made of each polysaccharide. Solutions were stored in a refrigerator to avoid the need for preservative, which could alter the performance of the polysaccharide films. To assess film strength, 15 mL of the 1% solutions were filled into rectangular silicone molds. The molds were placed in a humidity chamber set at 60 degrees C. and 20% relative humidity for one hour. At the end of one hour, the temperature was reduced to 50 degrees C. and 20% relative humidity. The solutions continued to dry overnight (˜12 hours) until the water completely evaporated, leaving a solid film. Films were then removed from the silicone molds and evaluated. See FIGS. 1-4.

In the case of many polysaccharides, the films were extremely brittle. The arabinoxylan alone as well as the flax (blend of arabinoxylan and pectin) were so brittle that the films could not be removed from the molds without breaking. Only a few (chitosan, hydroxypropyl methylcellulose, pectin, pullulan, and hyaluronic acid) provided strong films that can be pulled without breaking or tearing. Of these five polysaccharides, pullulan and chitosan both provided very thick, rigid films. Contrastingly, hydroxypropyl methylcellulose and hyaluronic acid provided more flexible films, similar to Saran Wrap™. Taking into account the other attributes (hydrophobicity and elasticity), we chose to combine chitosan and hydroxypropyl methylcellulose to give a film with optimal strength and flexibility, with the goal of improving hair strength while maintaining a natural feel on hair.

### Example 2. Methods for Assessing Hair Damage

The hair fiber is comprised of overlapping cuticle cells, which protect the inner corticular cells from mechanical stress from styling and heat exposure as well as chemical stress from bleaching and UV exposure. These cuticle cells tend to lift and can ultimately become damaged as the hair is subjected to everyday grooming habits such as shampooing and or styling the hair. These lifted cuticles can influence the interaction with light and are amenable to study with a light microscope.

As a result, the improvement of hair strength was evaluated as follows.

Virgin and Bleached hair swatches were washed before use using a sink with controlled flow and temperature (4 L/min at 35° C.). Hair was washed with a 14% aqueous solution of sodium laureth sulfate (SLES).

Images of the hair swatches were acquired using the Sensofar S Neox model. The microscope was set to take a standard light image, with the light source set at 25%, and magnification set to 50×.

Individual strands of hair were loaded onto a strain rig which fits on the microscope's Z-platform, allowing real-time imaging and strain application to the fibre. Images were taken at increasing increments of 7% strain. The number of lifted cuticles were quantified at each strain increment.

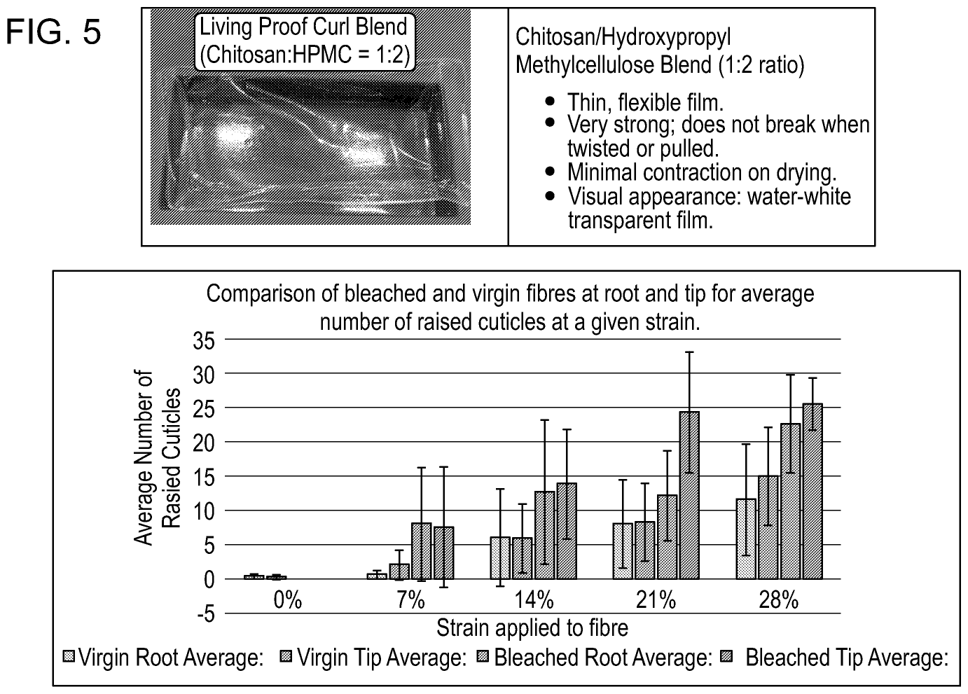

When subjected to increasing strain, the damaged hair represented by the bleached fibers showed an increase in the number of raised cuticles compared to the virgin hair fibers. See FIG. 5.

### Example 3. Improvement in Strength Using Inventive Composition

Aqueous Polysaccharide Solution

| Supplier | INCI Name | Trade Name | Qty | % W/W |
|---|---|---|---|---|
| N/A | Water/Aqua/Eau | Water, DI - room | 33.400000 g | 33.400000% |
| | | temperature | | |
| ChiBio Biotech | Chitosan | Water Soluble | 0.200000 g | 0.200000% |
| | | Mushroom Chitosan | | |
| Ashland Specialty | Hydroxypropyl | Benecel K35M | 0.400000 g | 0.400000% |
| Ingredients G.P. | Methylcellulose | | | |
| N/A | Water/Aqua/Eau | Water, DI - 5° C. | 66.000000 g | 66.000000% |
| | | Total | 100.000000 g | 100.00% |

Charge vessel with room temperature deionized water. Begin mixing at moderate speed. Add Chitosan to vortex. Mix for 15 minutes or until dissolved. Begin heating to 80-85° C. When the temperature of the batch is above 80° C., add Hydroxypropyl Methylcellulose to the vortex. Mix for not more than 10 minutes to disperse. Add chilled deionized water to the batch. Mix until Hydroxypropyl Methylcellulose is fully dissolved.

Bleached hair swatches were washed before use using a sink with controlled flow and temperature (4 L/min at 35° C.). Hair was washed with a 14% aqueous solution of sodium laureth sulfate (SLES). Half of the bleached hair swatches were than treated with the prepared aqueous polysaccharide solution. The treated swatches were prepared by submerging the bleached swatches into a beaker containing the above polysaccharide solution for a total of 5 seconds. Excess product was rinsed from the hair swatch for 30 seconds using a sink with controlled flow and temperature (4 L/min at 35° C.).

Images of both the bleached untreated and bleached treated hair swatches were acquired using the Sensofar S Neox model. The microscope was set to take a standard light image, with the light source set at 25%, and magnification set to 50×. The number of lifted cuticles were quantified at each strain increment.

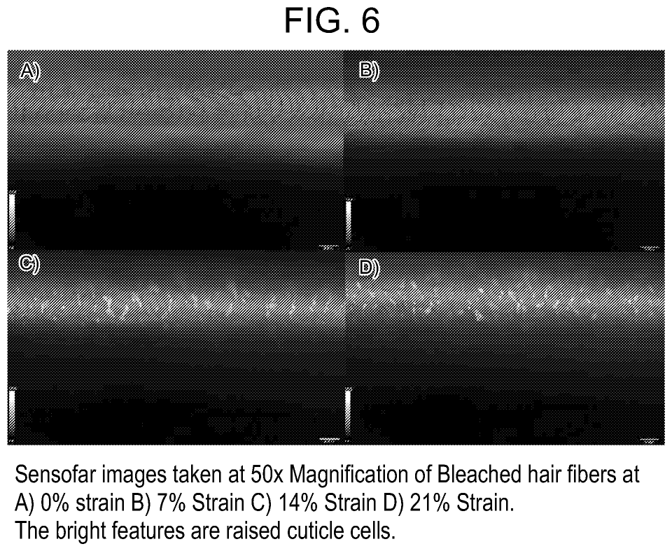

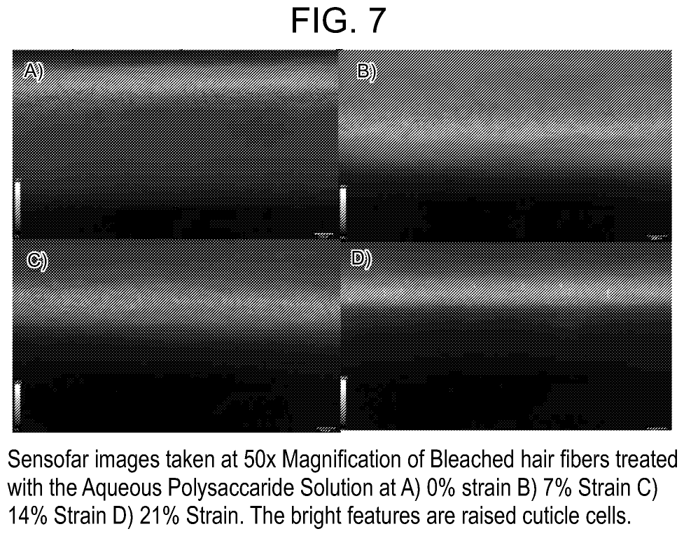

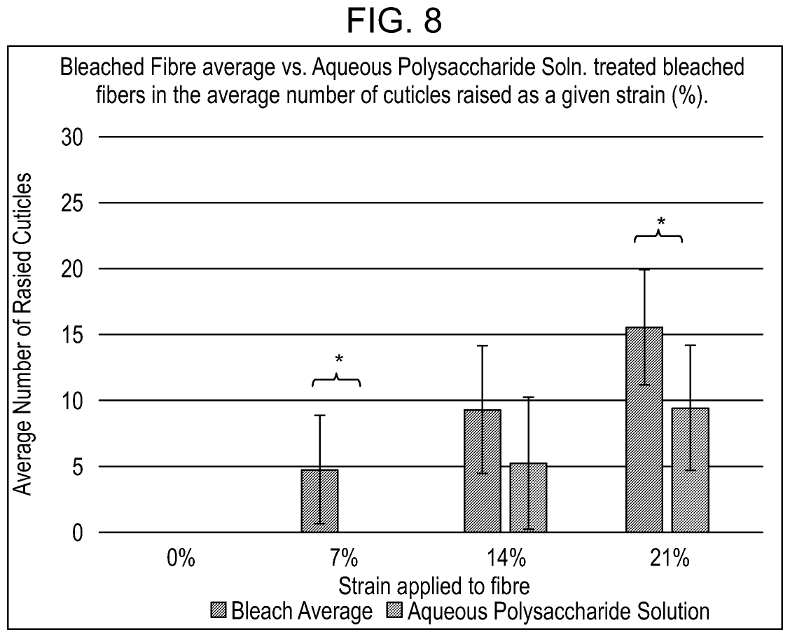

When subjected to increasing strain, the bleached hair swatches treated with the aqueous solution showed a decrease in the number of raised cuticles compared to the bleached hair fibers at lower levels of strain. represented by the bleached fibers showed an increase in the number of raised cuticles compared to the virgin hair fibers. See FIGS. 6-8.

### Example 4. Inventive Compositions

Shampoo

| Supplier | INCI Name | Trade Name | Qty | % W/W |
|---|---|---|---|---|
| N/A | Water/Aqua/Eau | Water, DI | 46.4350000 g | 46.4350000% |
| Lubrizol | Acrylates/C10-30 Alkyl | Carbopol Ultrez | 0.5000000 g | 0.5000000% |
| | Acrylate Crosspolymer | 21 Polymer | | |
| Innospec | Trisodium Ethylenedi- | Natrlquest E- | 0.5000000 g | 0.5000000% |
| | amine Disuccinate, | 30 | | |
| | Water/Aqua/Eau | | | |
| Innospec | Sodium Lauroyl Methyl | Iselux | 7.1580000 g | 7.1580000% |
| | Isethionate, Sodium | | | |
| | Methyl Isethionate, | | | |
| | Sodium Laurate, Lactic | | | |
| | Acid | | | |
| Innospec | Water/Aqua/Eau, Sodium | Pureact WS | 9.2850000 g | 9.2850000% |
| | Methyl Cocoyl Taurate, | Conc | | |
| | Coconut Fatty Acid, | | | |
| | Sodium Chloride, Sodium | | | |
| | Methyltaurate | | | |
| CLARIANT | Water/Aqua/Eau, | GlucoTain | 2.0000000 g | 2.0000000% |
| CORPORATION | Sunfloweroyl | Sense | | |
| | Methylglucamide | | | |
| AJINOMOTO | Water/Aqua/Eau, Disodium | Amilite ET- | 6.0000000 g | 6.0000000% |
| | Cocoyl Glutamate, Sodium | CS-12 | | |
| | Cocoyl Glutamate ,Sodium | | | |
| | Cocoyl Threoninate | | | |
| COLONIAL | Water/Aqua/Eau, | ColaTeric | 8.9740000 g | 8.9740000% |
| CHEMICAL, INC. | Babassuamidopropyl | BBS | | |
| | Hydroxysultaine | | | |
| Stepan | Glyceryl Caprylate/ | Stepan-Mild | 1.0000000 g | 1.0000000% |
| | Caprate, Glycerin | GCC | | |
| Symrise | Hydroxyacetophenone | SymSave H | 0.5000000 g | 0.5000000% |
| | | #672096 | | |
| GATTEFOSSE | Jojoba Esters (and) | Definicire | 1.0000000 g | 1.0000000% |
| | Helianthus Annuus | | | |
| | (Sunflower) Seed Wax | | | |
| | (and) Polylgycerin-3 | | | |
| Dewolf Chemical, Inc. | Cetearyl Alcohol | Lanette O | 2.5250000 g | 2.5250000% |
| Koster Keunen | Cetyl Stearate | Kester Wax | 2.0000000 g | 2.0000000% |
| | | K-56 | | |
| Aak | PEG-75 Shea Butter | Lipex 102 | 0.5000000 g | 0.5000000% |
| | Glycerides | E-75 100% | | |
| PREMIER | Rosmarinus Officinalis | Premox R- | 0.1000000 g | 0.1000000% |
| SPECIALTIES INC | (Rosemary) Leaf Extract, | 3204 | | |
| | Helianthus Annuus | | | |
| | (Sunflower) Seed Oil | | | |
| Evonik | Bis-(Isostearoyl/Oleoyl | Varisoft EQ | 0.2500000 g | 0.2500000% |
| | Isopropyl) Dimonium | 100 | | |
| | Methosulfate | | | |
| Lubrizol | PPG-20 Methyl Glucose | Glucam P-20 | 0.5000000 g | 0.5000000% |
| | Ether Distearate | Distearate | | |
| | | Emollient | | |
| CLARIANT | Lauryl/Myristyl | Genadvance | 0.5000000 g | 0.5000000% |
| CORPORATION | Polyricinoleate, Glycerin | Hydra | | |
| Dsm Nutritional | Potassium Cetyl Phosphate | Amphisol K | 0.5000000 g | 0.5000000% |
| Products Llc | | | | |
| Lincoln Fine | Propanediol | LinPure | 1.0000000 g | 1.0000000% |
| Ingredients | | | | |
| Lubrizol | PEG-120 Methyl Glucose | Glucamate | 0.5000000 g | 0.5000000% |
| | Trioleate, Water/Aqua/ | VLT | | |
| | Eau, Propanediol | | | |
| Ashland Specialty | Hydroxypropyl | Benecel E4M | 0.1000000 g | 0.1000000% |
| Ingredients G.P. | Methylcellulose | | | |
| ChiBio Biotech | Chitosan | Water Soluble | 0.0500000 g | 0.0500000% |
| | | Mushroom | | |
| | | Chitosan | | |
| USP | Water/Aqua/Eau (and) | Sodium | 1.0000000 g | 1.0000000% |
| | Sodium Hydroxide | Hydroxide 10% | | |
| Lubrizol | Polyquaternium-47, | Merquat 2001 | 0.6000000 g | 0.6000000% |
| | Water/Aqua/Eau, Sodium | N Polymer | | |
| | Benzoate | | | |
| Lubrizol | Polyquaternium-22, | Merquat 295 | 0.2130000 g | 0.2130000% |
| | Water/Aqua/Eau | | | |
| COLONIAL | Water/Aqua/Eau, Sodium | ColaLipid GS | 2.0000000 g | 2.0000000% |
| CHEMICAL, INC. | Grapeseed Amidopropyl | | | |
| | PG-Dimonium Chloride | | | |
| | Phosphate | | | |
| DKSH NORTH | Water/Aqua/Eau, | MG-60 | 2.0000000 g | 2.0000000% |
| AMERICA INC | Maltooligosyl Glucoside, | | | |
| | Hydrogenated Starch | | | |
| | Hydrolysate | | | |
| BIOSIL | Water/Aqua/Eau, | Methiopeptide | 0.0100000 g | 0.0100000% |
| TECHNOLOGIES, INC. | Methylthiopropylamido | | | |
| | Acetyl Methionine, | | | |
| | Methylpropanediol | | | |
| Schulke | Ethylhexylglycerin | Sensiva SC 50 | 0.2500000 g | 0.2500000% |
| Sozio Inc | Fragrance (Parfum) | SZUS030844 | 0.8000000 g | 0.8000000% |
| Living Proof | Octafluoropentyl | AN101 | 0.5000000 g | 0.5000000% |
| | Methacrylate (OFPMA) | | | |
| USP | Water/Aqua/Eau (and) | Sodium | 0.7500000 g | 0.7500000% |
| | | Hydroxide 10% | | |
| | | Total | 100.000000 g | 100.00% |

Conditioner

| | | Trade | | |
|---|---|---|---|---|
| Supplier | INCI Name | Name | Qty | % W/W |
| N/A | Water/Aqua/Eau | Water, DI | 71.4680000 g | 71.4680000% |
| Ashland Specialty | Hydroxyethylcellulose, | Natrosol | 0.2500000 g | 0.2500000% |
| Ingredients G.P. | Disodium Phosphate, Sodium | 250 HR CS | | |
| | Phosphate, Polysorbate 60 | | | |
| Ashland Specialty | Guar Hydroxypropyltrimonium | N-Hance 3000 | 0.3500000 g | 0.3500000% |
| Ingredients G.P. | Chloride, Water/Aqua/Eau | | | |
| RITA Chemical | Lactic Acid | Ritalac LA | 0.1500000 g | 0.1500000% |
| Corporation | | | | |
| Symrise | Hydroxyacetophenone | SymSave H | 0.2500000 g | 0.2500000% |
| | | #672096 | | |
| COLONIAL | Water/Aqua/Eau, Sodium | ColaLipid GS | 1.2500000 g | 1.2500000% |
| CHEMICAL, INC. | Grapeseed Amidopropyl PG- | | | |
| | Dimonium Chloride Phosphate | | | |
| Dewolf Chemical, Inc. | Cetearyl Alcohol | Lanette O | 6.7050000 g | 6.7050000% |
| Croda | Behentrimonium Chloride, | Incroquat | 1.6670000 g | 1.6670000% |
| | Isopropyl Alcohol | Behenyl TMC- | | |
| | | 85-PA-(MH) | | |
| Koster Keunen | Cetyl Stearate | Kester Wax K-56 | 0.6500000 g | 0.6500000% |
| Aak | PEG-75 Shea Butter | Lipex 102 E-75 | 0.5000000 g | 0.5000000% |
| | Glycerides | 100% | | |
| GATTEFOSSE | Jojoba Esters (and) | Definicire | 2.0000000 g | 2.0000000% |
| | Helianthus Annuus | | | |
| | (Sunflower) Seed Wax | | | |
| | (and) Polylgycerin-3 | | | |
| Croda | Hydroxyethyl | Crodamine SC- | 0.7500000 g | 0.7500000% |
| | Diethylenetriamine | SS-(MH) | | |
| | Dioleamide/Palmitamide | | | |
| PREMIER | Butyrospermum Parkii | Shea Butter | 2.0000000 g | 2.0000000% |
| SPECIALTIES INC | (Shea) Butter | #421580 | | |
| PREMIER | Rosmarinus Officinalis | Premox R-3204 | 0.1000000 g | 0.1000000% |
| SPECIALTIES INC | (Rosemary) Leaf Extract, | | | |
| | Helianthus Annuus | | | |
| | (Sunflower) Seed Oil | | | |
| Alzo | Linoleamidopropyl | Necon LO-80 | 0.5000000 g | 0.5000000% |
| International Inc. | Dimethylamine Dimer | | | |
| | Dilinoleate | | | |
| Evonik | Bis-(Isostearoyl/Oleoyl | Varisoft EQ 100 | 0.7500000 g | 0.7500000% |
| | Isopropyl) Dimonium | | | |
| | Methosulfate | | | |
| Solvay Fluorides Llc | Glyceryl Triacetyl | Mackaderm AR 33 | 1.0000000 g | 1.0000000% |
| | Hydroxy stearate | | | |
| CLARIANT CORPORATION | Lauryl/Myristyl | Genadvance Hydra | 1.0000000 g | 1.0000000% |
| | Polyricinoleate, Glycerin | | | |
| Centerchem | Squalane | Neossance Squalane | 1.0000000 g | 1.0000000% |
| SEPPIC INC | C21-28 Alkane | Emosmart C28 | 2.0000000 g | 2.0000000% |
| Lincoln Fine | Propanediol | LinPure | 1.5000000 g | 1.5000000% |
| Ingredients | | | | |
| Ashland Specialty | Hydroxypropyl | Benecel K200M | 0.2000000 g | 0.2000000% |
| Ingredients G.P. | Methylcellulose | | | |
| ChiBio Biotech | Chitosan | Water Soluble | 0.1000000 g | 0.1000000% |
| | | Mushroom Chitosan | | |
| Vertellus | Cetylpyridinium Chloride, | Freshstat CPC-PG | 0.7500000 g | 0.7500000% |
| | Propylene Glycol | | | |
| BIOSIL | Water/Aqua/Eau, | Methiopeptide | 0.0100000 g | 0.0100000% |
| TECHNOLOGIES, INC. | Methylthiopropylamido | | | |
| | Acetyl Methionine, | | | |
| | Methylpropanediol | | | |
| AJINOMOTO | Water/Aqua/Eau, Sodium | Ajidew NL-50 | 0.2500000 g | 0.2500000% |
| | PCA | | | |
| Sozio Inc | Fragrance (Parfum) | SZUS030844 | 0.8000000 g | 0.8000000% |
| Living Proof | Octafluoropentyl | AN101 | 2.0000000 g | 2.0000000% |
| | Methacrylate (OFPMA) | | | |
| RITA Chemical | Lactic Acid | Ritalac LA | 0.0500000 g | 0.0500000% |
| | | Total | 100.000000 g | 100.0 |

Strengthening of hair products are frequently quantified as a reduction in fiber breakage during repeated grooming experiment. Twenty dark-brown European hair tresses measuring 10 inches in length and 5 grams by weight were bleached. Ten of the bleached hair swatches were washed before use using a sink with controlled flow and temperature (4 L/min at 35° C.). Hair was washed with a 14% aqueous solution of sodium laureth sulfate (SLES). The remaining ten hair swatches were treated by applying a 15% dosage by weight of the hair swatch (i.e. 0.75 g per 5 g swatch) of the shampoo, massaging through the hair, then rinsing using a sink with controlled flow and temperature as described above. The conditioner was subsequently applied using the same technique. Both the control and treated swatches were dried and allowed to equilibrate overnight at 22° C./60% relative humidity.

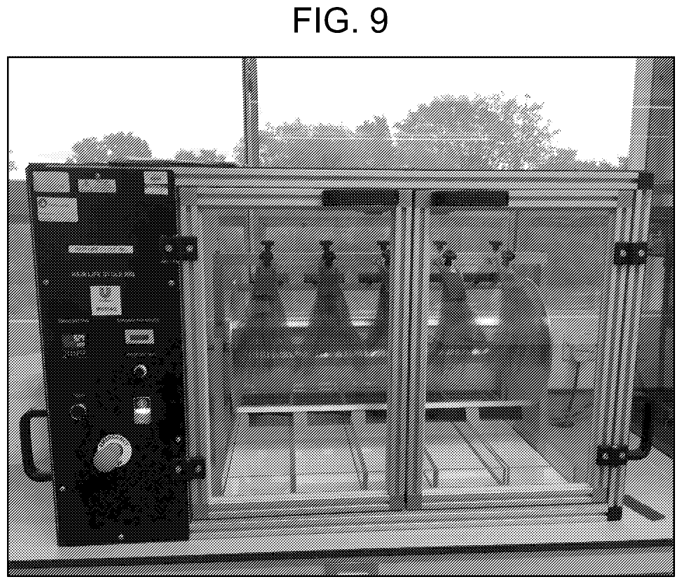

The dried swatches were loaded into a Lifecycle Rig set to one pass of the comb per second. See FIG. 9. Collection plates are position under each swatch to save the broken fiber fragments. Swatches were repeatedly brushed up to a total of 10,000 brush strokes. The quantity of broken fibers was recorded after 200, 600, 1,000, 2,000, 5,000, and 10,000 passes.

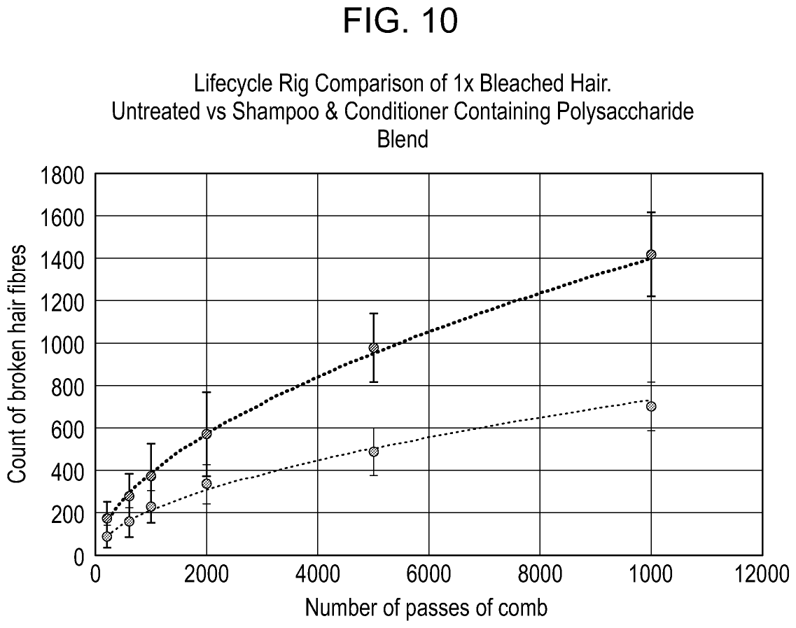

The inventive Shampoo and Conditioner demonstrate a significant reduction in fiber breakage compared to the swatches treated with the control. See FIG. 10.

### Example 5. Additional Inventive Compositions

Coily Styler

| Supplier | INCI Name | Trade Name | Qty | % W/W |
|---|---|---|---|---|
| N/A | Water/Aqua/Eau | Water, DI | 71.7850000 g | 71.7850000% |
| ChiBio Biotech | Chitosan | Water Soluble | 0.2000000 g | 0.2000000% |
| | | Mushroom Chitosan | | |
| Lincoln Fine | Propanediol | LinPure | 3.0000000 g | 3.0000000% |
| Ingredients | | | | |
| Ashland Specialty | Hydroxyethylcellulose, | Natrosol 250 | 0.7500000 g | 0.7500000% |
| Ingredients G.P. | Disodium Phosphate, Sodium | HR CS | | |
| | Phosphate, Polysorbate 60 | | | |
| Ashland Specialty | Guar Hydroxypropyltrimonium | N-Hance | 0.2000000 g | 0.2000000% |
| Ingredients G.P. | Chloride, Acrylamidopropyl- | 4572 | | |
| | trimonium Chloride/Acrylamide | | | |
| | Copoplymer | | | |
| RITA Chemical | Lactic Acid | Ritalac LA | 0.0500000 g | 0.0500000% |
| Corporation | | | | |
| Croda | Quaternium-91, PPG-3 | Crodazosoft | 1.5000000 g | 1.5000000% |
| | Benzyl Ether Myristate | SCQ-PA-(MH) | | |
| Croda | Behentrimonium Chloride, | Incroquat Behenyl | 2.0000000 g | 2.0000000% |
| | Isopropyl Alcohol | TMC-85-PA-(MH) | | |
| COLONIAL CHEMICAL, | Water/Aqua/Eau, Sodium | ColaLipid GS | 1.0000000 g | 1.0000000% |
| INC. | Grapeseed Amidopropyl PG- | | | |
| | Dimonium Chloride Phosphate | | | |
| Alzo | Linoleamidopropyl | Necon LO-80 | 0.0800000 g | 0.0800000% |
| International Inc. | Dimethylamine Dimer | | | |
| | Dilinoleate | | | |
| AJINOMOTO | PCA Ethyl Cocoyl | CAE | 0.2000000 g | 0.2000000% |
| | Arginate | | | |
| AJINOMOTO | Phytosteryl/Octyldodecyl | Eldew PS-203R | 0.2000000 g | 0.2000000% |
| SOLABIA USA | Behenyl/Stearyl | Rep'Hair | 0.3750000 g | 0.3750000% |
| INC | Aminopropanediol Esters | | | |
| Croda | Hydroxyethyl | Crodamine | 0.5000000 g | 0.5000000% |
| | Diethylenetriamine Dioleamide/ | SC-SS-(MH) | | |
| | Palmitamide | | | |
| Dewolf | Myristyl Alcohol | Lanette 14 | 2.5000000 g | 2.5000000% |
| Chemical, Inc. | | | | |
| Dewolf | Cetearyl Alcohol | Lanette 0 | 2.0000000 g | 2.0000000% |
| Chemical, Inc. | | | | |
| GATTEFOSSE | Jojoba Esters (and) | Definicire | 1.6000000 g | 1.6000000% |
| | Helianthus Annuus | | | |
| | (Sunflower) Seed Wax | | | |
| | (and) Polylgycerin-3 | | | |
| PREMIER | Butyrospermum Parkii | Shea Butter #421580 | 1.6000000 g | 1.6000000% |
| SPECIALTIES INC | (Shea) Butter | | | |
| Centerchem | Squalane | Neossance Squalane | 2.6000000 g | 2.6000000% |
| JEEN INTERNATIONAL | Ethylhexyl Stearate | Jeechem OS | 2.2500000 g | 2.2500000% |
| Inolex Chemical | Propylene Glycol | LexFeel Shine | 2.0000000 g | 2.0000000% |
| Company | Dibenzoate | | | |
| Ashland Specialty | Hydroxypropyl | Benecel K35M | 0.4000000 g | 0.4000000% |
| Ingredients G.P. | Methylcellulose | | | |
| Living Proof | Octafluoropentyl | AN101 | 2.0000000 g | 2.0000000% |
| | Methacrylate (OFPMA) | | | |
| Sozio Inc | Fragrance (Parfum) | SZUS030844 | 1.0000000 g | 1.0000000% |
| BIOSIL | Water/Aqua/Eau, | Methiopeptide | 0.0100000 g | 0.0100000% |
| TECHNOLOGIES, INC. | Methylthiopropylamido | | | |
| | Acetyl Methionine, | | | |
| | Methylpropanediol | | | |
| RITA Chemical | Lactic Acid | Ritalac LA | 0.2000000 g | 0.2000000% |
| Corporation | | | | |
| | | Total | 100.000000 g | 100.00% |

Curly Styler

| Supplier | INCI Name | Trade Name | Qty | % W/W |
|---|---|---|---|---|
| N/A | Water/Aqua/Eau | Water, DI | 59.1850000 g | 59.1850000% |
| ChiBio Biotech | Chitosan | Water Soluble | 0.2000000 g | 0.2000000% |
| | | Mushroom Chitosan | | |
| Lincoln Fine | Propanediol | LinPure | 1.3250000 g | 1.3250000% |
| Ingredients | | | | |
| Ashland Specialty | Guar Hydroxypropyltrimonium | N-Hance 4572 | 0.1500000 g | 0.1500000% |
| Ingredients G.P. | Chloride, Acrylamidopropyl- | | | |
| | trimonium Chloride/Acrylamide | | | |
| | Copoplymer | | | |
| Ashland Specialty | Hydroxyethylcellulose, | Natrosol 250 | 0.3500000 g | 0.3500000% |
| Ingredients G.P. | Disodium Phosphate, Sodium | HR CS | | |
| | Phosphate, Polysorbate 60 | | | |
| N/A | Water/Aqua/Eau | Water, DI | 20.0000000 g | 20.0000000% |
| Ashland Specialty | Water/Eau/Aqua, VP/DMAPA | Styleze | 5.0000000 g | 5.0000000% |
| Ingredients G.P. | Acrylates Copolymer | CC-10 | | |
| Dewolf Chemical, Inc. | Myristyl Alcohol | Lanette 14 | 2.3000000 g | 2.3000000% |
| Dewolf Chemical, Inc. | Cetyl Alcohol | Lanette 16 | 1.5000000 g | 1.5000000% |
| Croda | Water/Eau/Aqua, Cetrimonium | Incroquat CTC- | 1.5380000 g | 1.5380000% |
| | Chloride | 30-LQ-(MH) | | |
| Vantage Specialty | Ricinoleamidopropyl | Lipoquat R | 0.2000000 g | 0.2000000% |
| Ingredients Inc | Ethyldimonium Ethosulfate | | | |
| COLONIAL CHEMICAL, | Water/Aqua/Eau, Sodium | ColaLipid | 0.2500000 g | 0.2500000% |
| INC. | Grapeseed Amidopropyl PG- | GS | | |
| | Dimonium Chloride Phosphate | | | |
| AJINOMOTO | PCA Ethyl Cocoyl Arginate | CAE | 0.2000000 g | 0.2000000% |
| GATTEFOSSE | Jojoba Esters (and) | Definicire | 0.5000000 g | 0.5000000% |
| | Helianthus Annuus | | | |
| | (Sunflower) Seed Wax | | | |
| | (and) Polylgycerin-3 | | | |
| SOLABIA USA INC | Behenyl/Stearyl Aminopropanediol | Rep'Hair | 0.1500000 g | 0.1500000% |
| | Esters | | | |
| PREMIER | Butyrospermum Parkii | Shea Butter | 0.5000000 g | 0.5000000% |
| SPECIALTIES INC | (Shea) Butter | #421580 | | |
| Ashland Specialty | DimethylPABAmidopropyl Laurdimonium | Escalol HP | 0.0300000 g | 0.0300000% |
| Ingredients G.P. | Tosylate | | | |
| Inolex Chemical | Brassica Alcohol, Brassicyl | AminoSensyl HC | 1.3670000 g | 1.3670000% |
| Company | Valinate Esylate | | | |
| GATTEFOSSE | Glyceryl Behenate | Compritol 888 | 0.0600000 g | 0.0600000% |
| Croda | Steareth-21 | SP Brij S721 | 0.0500000 g | 0.0500000% |
| | | MBAL- SO-(AP) | | |
| Inolex Chemical | Propylene Glycol Dibenzoate | LexFeel Shine | 1.1000000 g | 1.1000000% |
| Company | | | | |
| Centerchem | Squalane | Neossance | 0.5000000 g | 0.5000000% |
| | | Squalane | | |
| JEEN INTERNATIONAL | Ethylhexyl Stearate | Jeechem OS | 0.5000000 g | 0.5000000% |
| Living Proof | Octafluoropentyl Methacrylate | AN101 | 1.2500000 g | 1.2500000% |
| | (OFPMA) | | | |
| SEPPIC INC | C13-15 Alkane | Emosmart L15 | 0.3500000 g | 0.3500000% |
| Croda | Polyamide-8 | SP OleocraftLP- | 0.2250000 g | 0.2250000% |
| | | 20-MBAL - PA-(MV) | | |
| Ashland Specialty | Hydroxypropyl Methylcellulose | Benecel E10M | 0.1000000 g | 0.1000000% |
| Ingredients G.P. | | | | |
| Ashland Specialty | Hydroxypropyl Methylcellulose | Benecel K35M | 0.3000000 g | 0.3000000% |
| Ingredients G.P. | | | | |
| BIOSIL | Water/Aqua/Eau, | Methiopeptide | 0.0100000 g | 0.0100000% |
| TECHNOLOGIES, INC. | Methylthiopropylamido | | | |
| | Acetyl Methionine, | | | |
| | Methylpropanediol | | | |
| Sozio Inc | Fragrance (Parfum) | SZUS030844 | 0.8000000 g | 0.8000000% |
| USP | Citric Acid | Citric Acid | 0.0100000 g | 0.0100000% |
| | | Total | 100.000000 g | 100.00% |

Wavy Styler

| Supplier | INCI Name | Trade Name | Qty | % W/W |
|---|---|---|---|---|
| N/A | Water/Aqua/Eau | Water, DI | 86.2680000 g | 86.2680000% |
| ChiBio Biotech | Chitosan | Water Soluble | 0.3750000 g | 0.3750000% |
| | | Mushroom Chitosan | | |
| Symrise | Pentylene Glycol | Hydrolite-5 | 0.7650000 g | 0.7650000% |
| Ashland Specialty | Hydroxyethylcellulose, Disodium | Natrosol 250 | 0.3000000 g | 0.3000000% |
| Ingredients G.P. | Phosphate, Sodium Phosphate, | HR CS | | |
| | Polysorbate 60 | | | |
| Dewolf Chemical, Inc. | Myristyl Alcohol | Lanette 14 | 2.0000000 g | 2.0000000% |
| Dewolf Chemical, Inc. | Cetyl Alcohol | Lanette 16 | 0.5000000 g | 0.5000000% |
| Croda | Water/Eau/Aqua, Cetrimonium | Incroquat CTC- | 1.5000000 g | 1.5000000% |
| | Chloride | 30-LQ-(MH) | | |
| Croda | Cetearyl Alcohol, Behentrimonium | SP Incroquat Behenyl | 0.6000000 g | 0.6000000% |
| | Chloride | TMC-25-MBAL-PA-(MH) | | |
| AJINOMOTO | PCA Ethyl Cocoyl Arginate | CAE | 0.2000000 g | 0.2000000% |
| GATTEFOSSE | Jojoba Esters (and) | Definicire | 0.0200000 g | 0.0200000% |
| | Helianthus Annuus | | | |
| | (Sunflower) Seed Wax | | | |
| | (and) Polylgycerin-3 | | | |
| SOLABIA USA INC | Behenyl/Stearyl Aminopropanediol | Rep'Hair | 0.0570000 g | 0.0570000% |
| | Esters | | | |
| Ashland Specialty | DimethylPABAmidopropyl | Escalol HP | 0.0250000 g | 0.0250000% |
| Ingredients G.P. | Laurdimonium Tosylate | | | |
| GATTEFOSSE | Glyceryl Behenate | Compritol 888 | 0.1000000 g | 0.1000000% |
| Croda | Steareth-2 | SP Brij S2 | 0.1500000 g | 0.1500000% |
| | | MBAL-SO-(AP) | | |
| Croda | Steareth-21 | SP Brij S721 | 0.1500000 g | 0.1500000% |
| | | MBAL-SO-(AP) | | |
| Inolex Chemical | Propylene Glycol Dibenzoate | LexFeel Shine | 1.6000000 g | 1.6000000% |
| Company | | | | |
| Living Proof | Octafluoropentyl Methacrylate | AN101 | 0.5000000 g | 0.5000000% |
| | (OFPMA) | | | |
| SEPPIC INC | C13-15 Alkane | Emosmart L15 | 0.4590000 g | 0.4590000% |
| Croda | Polyamide-8 | SP Oleocraft LP- | 0.1000000 g | 0.1000000% |
| | | 20-MBAL-PA-(MV) | | |
| Ashland Specialty | Hydroxypropyl Methylcellulose | Benecel E10M | 0.1000000 g | 0.1000000% |
| Ingredients G.P. | | | | |
| Ashland Specialty | Hydroxypropyl Methylcellulose | Benecel K200M | 0.3000000 g | 0.3000000% |
| Ingredients G.P. | | | | |
| Sozio Inc | Fragrance (Parfum) | SZUS030844 | 0.3200000 g | 0.3200000% |
| BIOSIL | Water/Aqua/Eau, | Methiopeptide | 0.0100000 g | 0.0100000% |
| TECHNOLOGIES, INC. | Methylthiopropylamido | | | |
| | Acetyl Methionine, | | | |
| | Methylpropanediol | | | |
| Akzo Nobel | Water/Aqua/Eau, Maltodextrin/ | BioStyle CGP | 1.0000000 g | 1.0000000% |
| | VP Copolymer, Maltodextrin, | | | |
| | PVP, 1-vinyl-2-pyrrolidone, | | | |
| | Phenoxyethanol, | | | |
| | Ethylhexylglycerin | | | |
| Croda | Water, Polyquaternium-72 | Mirustyle CP-LQ-(WD) | 2.6000000 g | 2.6000000% |
| USP | Citric Acid | Citric Acid | 0.0010000 g | 0.0010000% |
| | | Total | 100.000000 g | 100.00% |

Unless otherwise defined, all technical and scientific terms used herein are accorded the meaning commonly known to one with ordinary skill in the art.

1. A hair treatment composition comprising chitosan and hydroxypropyl methylcellulose (HPMC).

2. The hair treatment composition of claim 1, wherein the wt % ratio of chitosan to HPMC is about 1:2.

3. The hair treatment composition of claim 1, further comprising a cosmetically acceptable aqueous medium.

4. The hair treatment composition of claim 1, further comprising at least one product having a water content of 40% or greater.

5. The hair treatment composition of claim 1, wherein the hair treatment composition comprises at least one of a shampoo, a rinse-off conditioner, a leave-on conditioner, or a styling product.

6. The hair treatment composition of claim 1, wherein the hair treatment further comprise one or more additional ingredients selected from anionic surfactants, nonionic surfactants, amphoteric surfactants, cationic surfactants, fatty alcohols, fatty acids, emulsifiers, emollients, glycols, polymers or rheology modifiers, pH adjusters, UV absorbers, fragrance or other odor masking agents, and preservatives.

7. A method of improving the strength of hair comprising applying to the hair the composition of claim 1.
